(19) United States
(12) Patent Application Publication
(10) Pub. No.: US 2017/0071500 A1
(43) Pub. Date: Mar. 16, 2017

(54) ACQUISITION DEVICE FOR ELECTRICAL IMPEDANCE ANALYSIS

(71) Applicant: seca ag, Reinach BL (CH)

(72) Inventors: Marc-Oliver Von Maydell, Hamburg (DE); Wjatscheslaw Galjan, Nützen (DE)

(73) Assignee: seca ag, Reinach BL (CH)

(21) Appl. No.: 15/310,472

(22) PCT Filed: Mar. 9, 2015
(86) PCT No.: PCT/EP2015/054820

§ 371 Date: Nov. 11, 2016

(30) Foreign Application Priority Data
Jun. 2, 2014 (DE) 10 2014 107 718.0

(51) Int. Cl. A61B 5/053; A61B 5/04; A61B 5/0402; A61B 5/00

(52) U.S. Cl. CPC A61B 5/0537; A61B 5/7246; A61B 5/04011; A61B 5/0402; A61B 5/4869; A61B 5/6825; A61B 5/6829; A61B 5/7221

(57) ABSTRACT

An acquisition device for electrical impedance analysis for determining body composition parameters includes a control and analysis unit including measuring and processing circuitry and at least two electric lines, each of which lines is connected to the control and analysis unit and has an electrode. Each electrode is dedicated for connection at an assigned measuring position out of a set of predetermined measuring positions. The control and analysis unit is arranged to cause application of alternating current through electrodes and to measure a resulting voltage between electrodes; record a time dependent electric signal generated by cardiac activity of the subject for each pair of electrodes; and by comparing the time dependent electric signals with expected electric signals of cardiac activity for the predetermined measuring positions, assign to the electrodes those measuring positions for which the time dependent electric signals best fit the expected electric signal of the cardiac activity.

The present invention is directed to an acquisition device for electrical impedance analysis for determining body composition parameters of a body of a subject, comprising a control and analysis unit including measuring and processing circuitry and at least two electric lines, each of which lines being connected to the control and analysis unit and having an electrode, each electrode being dedicated for connection at an assigned measuring position out of a predetermined set of measuring positions, which predetermined measuring positions are suitable for electrical impedance analysis of a human body, wherein the control and analysis unit is arranged to cause application of alternating current through electrodes and to measure a resulting voltage between electrodes.

The conductivity of the human body strongly depends on the water content. Since the fat-free portions of the body, such as muscles and body fluids, contain a major part of the water of the body, whereas fat tissue contains a very low water content because of its hydrophobic properties, conclusions can be drawn on the relative amount of fat by determining the conductivity of the body or of a body segment (or by determining the resistance or impedance), wherein further body related data such as height and body weight of the subject examined are taken into a consideration in the determination.

As body composition parameters to be determined in particular characteristic values of the body representative for the fat content are derived from the impedance measurements, wherein as values representative for the fat content are meant to cover not only the fat content of the body itself, but also characteristic body values proportional or complementary to the fat content, for example the fat-free mass (PPM) of the body (which is complementary to the fat mass), the fat mass (FM) the total body water mass (TBW) extracellular water mass (ECW), or the lean soft tissue mass (Lean-Soft-Tissue, LST).

An exemplary acquisition device for electric impedance analysis for determining the fat content of a human body is described in WO 91/01303. The device described has eight electrodes, which are connected by eight lines to a control and analysis unit. These electrodes include four foot electrodes which are located on a platform wherein the subject to be examined steps on this platform. In addition, four hand electrodes are provided on two handles, which handles have to be grasped by the subject to be examined with his/her two hands. An alternating current is then injected via respectively two electrodes located on different limbs, and the voltage is measured at two electrodes, likewise in contact with different limbs. By transitioning to other pairs of current injecting electrodes and voltage measuring electrodes different measuring programs can be carried out successively and different body segments can be examined successively. Furthermore, an entire body side can be measured in the case of feeding of current into a hand and a foot and measuring voltage on the same hand and on the same foot. There are also devices in which current is injected and voltage is measured simultaneously using a single electrode pair; in such a cases the part of the circuit external to the body of the subject to be examined has to be taken into account with its resistance, and the contribution of the external circuit part outside of the body has to be corrected for when determining the resistance or impedance of the body. Therefore, in principle it is possible to use a device with at least two lines, each line being provided with an electrode, to acquire signals for electrical impedance analysis of the human body.

The technical background and a variety of bioimpedance measurements and their use for determination of body composition parameters are for example described in the article entitled “Bioelectrical impedance analysis—part I: review of principles and methods” by Ursula G. Kyle et al., Clinical Nutrition (2004) 23, pages 1226-1243. The methods and devices are based on different function principles. There are simple devices having only two or four electrodes, which are for example connected to one hand and one foot on one side of the body, wherein in this manner the impedance of a body half is integrally determined. With other devices as described above impedance values of single body segments can be determined. Furthermore, there are devices which operate using a single frequency for the alternating current, for example 50 kHz. In addition, there are so called multi-frequency bioimpedance analysis methods which utilize a series of measuring frequencies, for example measuring frequencies of 0.1, 5, 50, 100, 200 to 500 kHz. These analysis methods are rather sensitive to differentiate between extracellular water and intracellular water. In the article mentioned above a number of regression equations are described, which equations described a body composition value as a sum of personnel parameters (age, sex, etc.) and impedance values, wherein each term in the sums is provided with an empirical coefficient. These coefficients are determined in statistical studies in which for a representative group of subjects the values of the body composition parameter are determined by independent methods, and impedance measurements are performed in addition. The coefficients are calculated such that the best correlation between the independently determined body composition parameters and the body composition parameters determined based on the impedance value is obtained.

Further information on devices for bioimpedance measurement and for bioimpedance analysis methods are described in the article “Whole-body impedance—what does it measure?” by Kenneth R. Foster et al., Am. J. Clin, Nutr. 1996, 64 (suppl): pages 388-396.

There are, as described above, signal acquisition devices having a standing platform and handles, which signal acquisition devices are configured to be able to examine persons while standing upright; besides that there are devices that are suitable for lying or sitting persons. A device for bioimpedance measurement of a lying subject is for example described in US 2013/0102873 A1 which is the basis for the preamble of claim 1. The device comprises a central unit which is placed beside a bed on which a subject lies. From the central unit lines extend which carry electrodes at their ends, which electrodes are dedicated for connection to measuring positions on the left hand and on the right hand as well as on the left foot and on the right foot of the subject. The lines are sufficiently long such that they can, starting from the central unit, be placed on the subject and can be laid to the dedicated measuring positions on the hands and feet of the subject. In such devices it must either be specified from the very beginning which cable is dedicated to which measuring position, or information has to be entered after the electrodes are connected which electrode is connected to which measuring position. This means on the one hand effort for the operating personnel, and on the other hand can lead to wrong results in case of data input mistakes such that the measurement has to be repeated after correction of the input.

It is an object of the present invention to provide an acquisition device for electrical impedance analysis, which device may be connected to a subject in a simple manner and which is easy to operate.

This object is achieved by an acquisition device for electrical impedance analysis comprising the features of claim 1, preferred embodiments of the invention are set out in the dependent claims.

According to the invention the control and analysis unit is arranged to detect between each pair of two electrodes a time dependent voltage signal caused by cardiac activity of a subject, and, by comparison of the detected signal with signals of cardiac activity expected for the measuring positions out of the predetermined set of measuring positions, to assign to the electrodes those measuring positions out of the predetermined set of measuring positions for which the detected time dependent electrical signal fit best to the electrical signal of cardiac activity to be expected for these measuring positions. In this manner it can be determined automatically by the control and analysis unit which electrode is in contact at which measuring position out of the predetermined set of measuring positions. Once for each electrode its measuring position is assigned, different measuring programs can be carried out based on this information as will be explained further below.

The predetermined set of measuring positions may for example comprise following measuring positions: right hand (RA), right foot (RL), left hand (LA), and left foot (LL). If electrodes are connected to four measuring positions detected signals of all electrode pairs are compared to the expected cardiac activity signals for all possible pairings of measuring positions (RA-RL; RA-LA, RA-LL, RL-RA, RL-LA, . . . ), and measuring positions are assigned to the electrodes such that best agreement between expected and detected cardiac activity signals is obtained.

The invention is based on the fact that, besides alternating current and voltage signals for the impedance measurement, in addition a low-frequency voltage signal according to the principle of electrocardiograms is detected. Since in case of the “normal” electrocardiogram recoding of potentials, the R-wave of the cardiac potential has a positive voltage, the actual orientation of the patient and the locations of the measuring electrodes, e.g. on hands and feet, can be recognized by analyzing the polarity of the R-wave. If the acquisition device is disposed with respect to the subject such that a positive R-weave means that the head of the patient is lying on the left hand side, and therefore the lines laid to the left hand side are leading to the hands, an actually detected negative R-wave would mean that the head is actually lying on the right hand side and that the lines leading to the right hand side are connected to the measuring positions on the hands. If the orientation of the patient and the assignment of the electrodes to the measuring positions on the body of the patient are known, the control and analysis unit can process the data accordingly such that for the determined measuring positions of the electrodes correct formulas for calculating the body composition parameter are utilized in a correct manner.

With the device according to the invention it is no longer necessary to pay attention which electrode is connected to which measuring position or no input is necessary by the operating personnel on the assignment of the electrodes to the measuring positions since this assignment is automatically detected.

A further disadvantage of the prior art according to US 2013/0102873 A1 is that the lines from the remotely disposed central unit have to be laid first to the bed and then to be laid on the subject to the respective measuring positions. Furthermore, stowage of the cables after a measuring operation is time-consuming for the personnel because the relatively long cables for example have to be wound up.

In a preferred embodiment a flat, extensive cover pad body is present which is flexible at least in portions thereof to be placed and rest on the body of a subject, which cover pad may for example be placed transversely over the abdomen or over the legs of the subject. From the cover pad body lines extend to the measuring positions. The lines extend into the interior of the cover pad body where also the control and analysis unit can be accommodated as a whole or in parts. The cover pad body can for example take the form of a flexible mat which may have an elongated rectangular shape and which may be put on with its elongated extension transversely over the subject. Four lines may for example be provided which are guided out of the rectangular mat in the areas of the corners thereof. After putting the cover pad body on the subject lines of relatively short length are sufficient to lay them to the intended measuring positions, for example to the hands and feet of the subject. After completion of a measurement the flexible mat may be roiled up and may thus be stored in a space saving configuration.

The cover pad body comprises for example a flexible mat of insulating material, with for example four cables leading out. Through each cable two lines extend, each carrying an electrode at its end. The four cables are dedicated for contacting the two hands and the two feet of the person to be examined. In principle it is also possible to have devices with only two such cables which are, intended to be connected to the limbs of one body side of a subject. Furthermore, it is in principle possible that only two lines, each with an electrode, are provided, the electrodes being designate to contact two limbs, wherein current injection and voltage measurement are performed on the same electrodes.

The control and analysis unit may be integrated in the flexible mat. In principle, however, it is also possible that only the lines and circuitry for selective current injection and for voltage measurement are accommodated in the mat, whereas control and analysis functions are implemented separately therefrom in a remotely disposed data processing unit capable of communicating with the acquisition device by a wire or wireless connection. At one end of the flexible mat in the form of a long sheet an operating unit including keys may be provided by which the device may be operated.

According to the characterizing features of claim 1 it is possible that the mat is put on the subject in one direction or in the opposite direction, i.e. it is not important whether for example the hands are on the left hand side and the feet oppositely on the right hand side thereof, or vice versa. In order to automatically determine the orientation the control and analysis unit is arranged to detect time dependent electrical signals generated by the cardiac activity of the subject in the electrode signals, and to compare the detected signals of cardiac activity with electrical signals of cardiac activity expected for the measuring positions of the predetermined set of measuring positions. The electrodes are then assigned to those measuring positions of the predetermined set of measuring positions for which the detected time dependent electrical signals best match the expected electrical signals of cardiac activity for these measuring positions. The predetermined set of measuring positions may for example include four measuring positions, namely left and right hand as well as left and right foot of the subject. With the acquisition device according to the present invention it is of no relevance on which side of the cover pad body the hands are located and on which side the feet are located, i.e. the cover pad body can be laid over the subject in one direction or turned by 180°.

This may for example be of important if subjects lying in beds are to be examined. If beds are standing with one side adjacent to a wall and if a person conducting the measurement steps next to the free side of a bed standing on a wall, the head of the subject to be examined can either be on the right hand side or on the left hand side of the mat put on the subject. Since the orientation of the mat may be dictated by the fact that an operating unit disposed on one end of an elongated mat has to be located on the accessible side of the bed, the system is flexible such that in this orientation one line is connected to a foot and one line to a hand of the person to be examined, wherein for both possible locations of the limbs relative to the mat (legs on the left hand sided and arms on the right hand side or legs on the right hand side and arms on the left hand side) a correct measurement is carried out. With the acquisition device according to the present invention it is not necessary to pay attention on which side of the cover pad body the hands allocated and on which side the feet of the subject are located. Furthermore, it is not required that the operating personnel provides any input regarding the orientation of the subject (head on the left hand side or on the right hand side of the cover pad body), since the control and analysis unit automatically assigns to the electrodes in contact the measuring positions selected from the predetermined set of measuring positions. This further simplifies operation of the acquisition device for the operating personnel and the risk of operational mistakes due to erroneous inputs and assignments of measuring positions of the electrodes is reduced.

In an advantageous embodiment the control and analysis unit is arranged to perform the electrical impedance analysis of the subject using measuring programs which are executable with the measuring positions found for the electrodes. If four example one measuring position on the left hand and one measuring position on the left foot of the subject are found, with such electrode configuration the impedance of one body side (the left body side) of the subject can be measured. If electrodes are found in measuring positions on both hands and both feet many measuring programs may be performed in addition, which measure the impedance of individual body segments, as will be described in more detail below.

Preferably the control and analysis unit is further arranged to offer such measuring programs for the electric impedance analysis of the subject which measuring programs can be executed with the measuring positions found for the electrodes, to the operating personnel for selection, and to execute the selected measuring programs.

In a preferred embodiment two double lines are connected to the control and analysis unit, each double line comprising one line with an electrode for injecting an alternating current and one line with an electrode for measuring voltage, wherein the predetermined set of measuring positions includes a measuring position on an arm, in particular on a hand, and a measuring position on a leg, in particular on a foot, of one body side of the subject, and wherein information on the electric signals of cardiac activity expected for these measuring positions are stored in the control and analysis unit.

It is further preferred that four double lines are connected to the control and analysis unit, each double, line comprising one line having an electrode for injecting an alternating current and one line with an electrode for measuring voltage, wherein the set of predetermined measuring positions includes four measuring positions on four extremities of the subject, and wherein information on the, electric signals of cardiac activity expected for these measuring positions are stored in the control and analysis unit. In particular, the set of predetermined measuring positions may include measuring positions on both hands and both feet of the subject.

In a preferred embodiment the control and analysis unit is further arranged to issue a warning if the electric signals of cardiac activity recorded by the electrodes deviate by more than a predetermined amount from the expected electric signals of cardiac activity for the measuring positions of the electrodes on the hands and feet of the subject, to thereby point out a mistake in the electrode placement.

Preferably the control and analysis unit is arranged to check whether an electrode is assigned to a measuring position on a hand, and an electrode is assigned to a measuring position on the foot of the subject on the same body side, and if this is the case, to execute a measuring program in which alternating current is injected through the electrodes on hand and foot and in which the resulting voltage between electrodes on the same hand and on the same foot are measured. Based on this the impedance of the body side which is contacted by the electrodes on hand and foot is determined.

Preferably, the control and analysis unit is arranged to check whether three electrodes are assigned to the left and right hand and to a foot, and if this is the case, to inject current through the electrodes on the hand and on the foot of the same body side and to determine the resulting voltage between the electrodes on the hands, to thereby determine the impedance of the arm through which current is flowing.

Preferably the control and analysis unit is arranged to check whether three measuring positions on both feet and on a hand are assigned to electrodes, and if this is the case, to execute a measuring program in which an alternating current is injected through a hand and a foot of the same body side, and in which the resulting voltage between the electrodes on the feet is determined, to thereby determine the impedance of the leg through which current flows.

Preferably the control and analysis unit is arranged to check whether four electrodes are assigned to measuring positions on both hand and both feet of the subject, and if this is the case, to execute a measuring program in which alternating current is injected through the electrodes on a hand and a foot on the same body side, and in which the resulting voltage between the electrodes on the hand and the foot on the opposite body side is determined, to thereby determine the impedance of the torso of the subject.

In the following the invention will be described with reference to an embodiment in the drawings, in which:

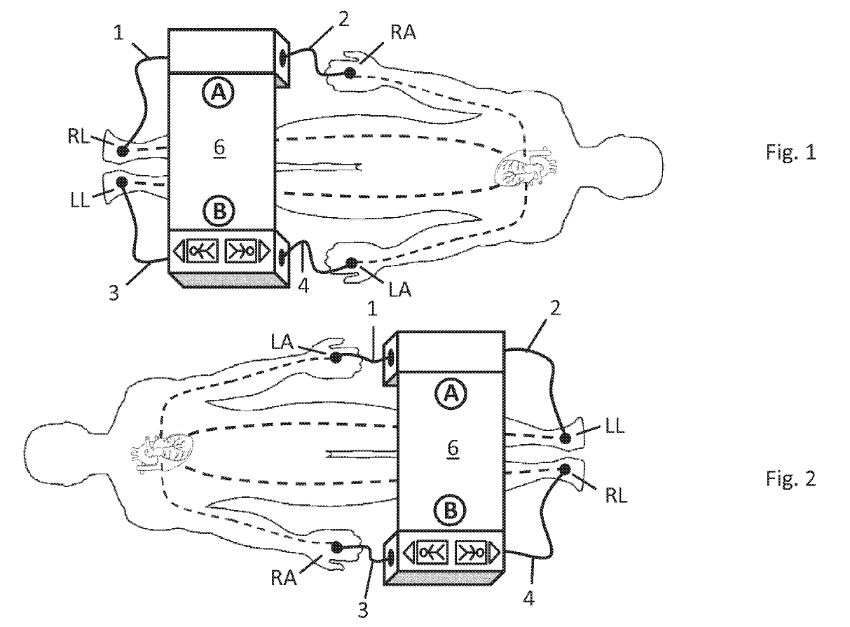

FIG. 1 is a schematic presentation of a lying subject and an acquisition device for electrical impedance analysis;

FIG. 2 is a corresponding view as in FIG. 1, wherein the subject is lying with respect to the acquisition device in an opposite orientation;

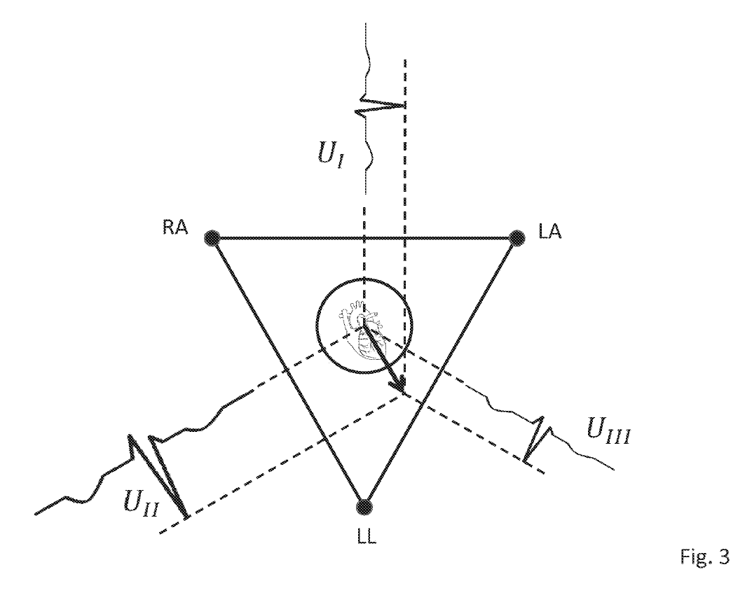

FIG. 3 is a schematic presentation of cardiac activity signals with ECG recordings on the right arm, on he left arm and on the left leg;

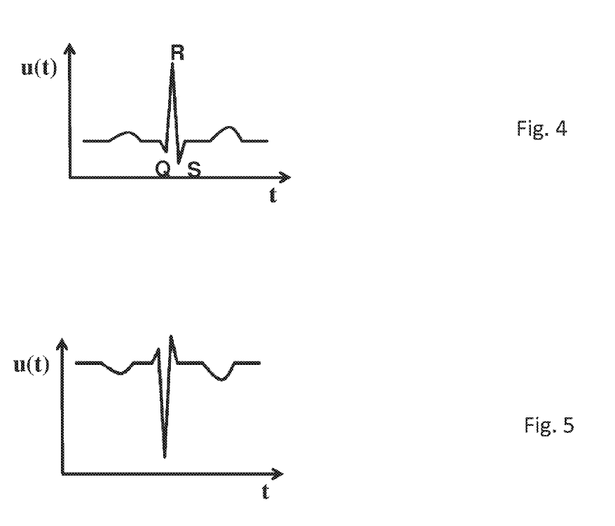

FIG. 4 schematically shows an electric cardiac activity signal in case of a first orientation of the subject with respect to the acquisition device and

FIG. 5 shows an electric cardiac activity signal of the subject in case of an opposite orientation of the subject with respect to the acquisition device and correspondingly oppositely connected electrodes.

FIG. 1 schematically shows a lying subject with an acquisition device laid on in the region of the knees, which acquisition device is provided in form of a flexible mat 6 having on both ends A and B electric circuits in respective housings, wherein at the end designated by B also operating keys are provided. From each of the two opposite ends two cables extend, namely the cables 1 and 2 from end A, and the cables 3 and 4 from end B. Each cable may contain two electric lines, each line, having an electrode at its end, wherein one electrode of a cable is used for current injection and the other electrode of the cable is used for voltage measurement.

As shown in FIG. 1 the electrodes are connected to the measuring positions right hand (RA), right foot (RL), left hand (LA), and left foot (LL).

In principle it is also possible that the subject lies in opposite orientation with respect to the acquisition device, as shown in FIG. 2. If in such case the acquisition device cannot be placed on the subject in opposite orientation, for example because there is a wall on the opposite side of the bed, now the electrodes from cable 1 are connected to position LA, the electrodes from cable 2 to position LL, the electrodes from cable 3 to measuring position RA, and the electrodes from cable 4 to measuring position RL.

In order to perform correct measuring programs and to analyze the results correctly, in principle it has to be known whether the subject lies in the orientation shown in FIG. 1 or in the orientation shown in FIG. 2.

According to the present invention the control and analysis unit in the acquisition device is arranged to derive from the electrodes electrical signals representing cardiac activity, and to compare these signal with expected cardiac signals for the predetermined set of measuring positions, and to assign such measuring positions to the electrodes such that the detected cardiac activity signals match the expected electrical cardiac activity signals in the best manner.

ECG recordings according to Einthoven are for example:
I=LA−RA
II=LL−RA
III=LL−LA,

FIG. 3 shows a schematical presentation of the signals for these ECG recordings.

If one assumes now for example that the end A of the acquisition device is connected to the right body side of the person, these results in the following connections

Cable 1=RL

Cable 2=RA

Cable 3=Lu

Cable 4=LA.

One can now with each of the three ECG recordings according to Einthoven check the correctness of this assumption. For example, first the recording I=LA−RA is checked, i.e. the voltage between electrodes of cables 2 and 4. This results in the electric cardiac activity signal shown in FIG. 4 having a positive R-wave, which confirms the assumption that the end A of the acquisition device is connected to the right body side of the subject.

On the other hand, if the recording I shows an electric cardiac activity signal as in FIG. 5, it has to be concluded that the assumption is incorrect and that the subject is indeed lying in opposite direction since the R-wave has the wrong polarity. The assumption can be checked with cables 1 and 3 for confirmation.

Corresponding checks of the assumption are possible with the recordings II and III.

The electrodes then have to be assigned to the measuring positions LA, RA, LL, and RL in such a manner that the recorded electric cardiac activity signals at the assigned measuring positions fit to the expected electric cardiac activity signals in the best manner. The average expected electric cardiac activity signals for the predetermined set of measuring positions LA, RA, LL, and RL may be available in a memory and may be compared to currently recorded signals. For the comparison a numerical measure of the agreement may be determined in each case, for example by allowing for a free factor and by providing the expected cardiac activity signals with scaling factors, wherein the factor and the scaling factor in each case are numerically matched by a fitting procedure such that for example the error square (sum of the error squares) between the recorded cardiac activity signal and the expected cardiac activity signals is minimized. The error square is then in each case a measure of how well the recorded cardiac activity signal matches the expected cardiac activity signals, wherein the best agreement is obtained for the signal pair having the least error square or standard deviation. Such numerical measures on the degree of agreement can be determined for all three recordings I, II, and III, and then the measures for the agreement may be combined in order to determine which electrode positions of the predetermined set of electrode positions has the highest likelihood to be correct. Alternatively only particular signal features may be considered, for example the polarity of the R-wave, and based on this the electrode positions may be assigned to the measuring positions of the set of predetermined measuring positions, wherein for plural recordings I, II, and III the most frequent assignment to the predetermined measuring positions LA, RA, LL, and RL is selected as the most likely, and is assigned to the respective electrode.

In principle it can be determined in an advance measurement how many and which electrodes have contact. Then for each pair of electrodes a cardiac activity signal is recorded and is then compared to the cardiac activity signals to be expected for the set of measuring positions RA, LA, RL, and LL between any pairs of electrodes at the these measuring positions, whereafter the measuring positions of the set RA, LA, RL, and LL are assigned to the electrodes in such a manner that the actually recorded signals fit to the expected cardiac activity signals in the best manner.

With the acquisition device according to the invention no data input regarding the orientation of the subject relative to the acquisition device is needed, since the orientation of the subject is derived from the signals recorded by the electrodes.

The control and analysis unit may be partly or completely integrated into the acquisition device shown in the figures, and after a measurement only the results are transmitted to a remotely disposed analysis and display unit. Alternatively parts of the functions of the control and analysis unit may be implemented in a separate data processing unit disposed remotely from the acquisition device shown in the figures, wherein data exchange between the acquisition device and the data processing unit may be performed through wire connections or wireless

1. Acquisition device for electrical impedance analysis for determining body composition parameters of a subject having a body, said acquisition device comprising:
a control and analysis unit including measuring and processing circuitry and at least two electric lines,
each of said lines being connected to the control and analysis unit and including an electrode,
each electrode being dedicated for connection at an assigned measuring position selected from a predetermined set of predetermined measuring positions, which predetermined measuring positions are suitable for electrical impedance analysis of the body,
wherein the control and analysis unit is configured to cause application of alternating current through an injection pair of said electrodes and to measure a resulting voltage between a measurement pair of said electrodes,
wherein the control and analysis unit is configured to, for each pair of said electrodes, acquire a time dependent electric signal generated by cardiac activity of the subject and, by comparing the time dependent electric signals with expected electric signal of cardiac activity to be expected for the predetermined measuring positions, to assign to each of the electrodes a respective one of the assigned measuring positions, selected from the predetermined set of measuring positions, for which the time dependent electric signal fits to the expected electric signal in the best manner.

2. Acquisition device according to claim 1,
wherein a flat, extensive cover pad body is provided which is at least in portions thereof flexible and which is configured to be placed on the body of the subject,
wherein the lines extend from the cover pad body to the measuring positions, and
wherein parts of or the whole control and analysis unit are contained in the cover pad body.

3. Acquisition device according to claim 1,
wherein the control and analysis unit is further configured to perform electrical impedance analysis for the subject,
wherein only such analyses are carried out which are executable with the assigned measuring positions determined for the electrodes.

4. Acquisition device according to claim 1,
wherein the control and analysis unit is further configured to offer to an operating personnel for selection such measuring programs for electrical impedance analysis of the subject which are executable with the assigned measuring positions found for the electrodes, and to execute the selected measuring programs.

5. Acquisition device according to claim 1,
wherein two double lines are connected to the control and analysis unit, each double line comprising a first line with a first electrode for injecting an alternating current and another line with another electrode for measuring voltage,
wherein the predetermined set of measuring positions includes for one body side of the subject a measuring position on an arm and a measuring position on a leg, and
wherein information on expected electric signals of cardiac activity expected for said measuring positions on an arm and on a leg are stored in the control and analysis unit.

6. Acquisition device according to claim 1,
wherein four double lines are connected to the control and analysis unit, each double line comprising a first line with a first electrode for injecting an alternating current and another line with another electrode for measuring voltage,
wherein the predetermined set of measuring positions includes four measuring positions on four extremities of the subject, and
wherein information on expected electric signals of cardiac activity expected for said four measuring positions of the four extremities is stored in the control and analysis unit.

7. Acquisition device according to claim 6,
wherein the predetermined set of measuring positions includes a measuring position on a right hand, a measuring position on a left hand, a measuring position on a right foot, and a measuring position on a left foot of the subject, and
wherein information on expected electric signals of cardiac activity expected for said measuring positions on a left hand, a right foot, and a left foot is stored in the control and analysis unit.

8. Acquisition device according to claim 7,
wherein the control and analysis unit is further configured to issue a warning if recorded electric signals of cardiac activity recorded by the electrodes deviate by more than a predetermined amount from the expected electric signals of cardiac activity for the assigned measuring positions of the electrodes on the hands and feet of the subject, to thereby point out a mistake in the electrode placement.

9. Acquisition device according to claim 1,
wherein the acquisition device is provided with a communication device for data exchange of measuring and control information with a further data processing unit.

10. Acquisition device according to claim 7,
wherein the control and analysis unit is configured to check whether one of said electrodes is assigned to a measuring position on a hand, and whether another of said electrodes is assigned to a measuring position on a foot of the subject on the same body side, and if this is the case, to execute a measuring program in which alternating current is injected through the electrodes on said hand and foot, and in which resulting voltages between electrodes on the same hand and on the same foot are measured,
wherein based on said resulting voltages the impedance of the body side which is contacted by the electrodes on hand and foot is determined.

11. Acquisition device according to claim 7,
wherein the control and analysis unit is configured to check whether three of said electrodes are respectively assigned to the left and right hand and to a foot, and if this is the case, to inject current through the electrodes on the hand and on the foot of the same body side, and to determine a resulting voltage between the electrodes on the hands, to thereby determine the impedance of the arm through which current is flowing.

12. Acquisition device according to claim 7,
wherein the control and analysis unit is configured to check whether a plurality of said electrodes are respectively assigned to three measuring positions on both feet and on a hand, and if this is the case, to execute a measuring program in which an alternating current is injected through a hand and a foot of the same body side, and in which a resulting voltage between the electrodes on the feet is determined, to thereby determine the impedance of the leg through which current flows.

13. Acquisition device according to claim 7,
wherein the control and analysis unit is configured to check whether four of said electrodes are respectively assigned to measuring positions on both hands and both feet of the subject, and if this is the case, to execute a measuring program in which an alternating current is injected through the electrodes on a hand and a foot on the same body side, and in which a resulting voltage between the electrodes on the hand and the foot on an opposite body side is determined, to thereby determine the impedance of a torso of the subject.

14. Acquisition device according to claim 1,
said injection pair of said electrodes and said measurement pair of said electrodes being the same.

15. Acquisition device according to claim 2,
wherein the control and analysis unit is further configured to perform electrical impedance analysis for the subject,
wherein only such analyses are carried out which are executable with the assigned measuring positions determined for the electrodes.

16. Acquisition device according to claim 2,
wherein the control and analysis unit is further configured to offer to an operating personnel for selection such measuring programs for electrical impedance analysis of the subject which are executable with the assigned measuring positions found for the electrodes, and to execute the selected measuring programs.
